(19) United States
(12) Patent Application Publication
(10) Pub. No.: US 2012/0201857 A1
(43) Pub. Date: Aug. 9, 2012

(54) Transdermal delivery system for therapeutics

(76) Inventor: Pankaj Modi, Ancaster (CA)

(21) Appl. No.: 12/661,453

(22) Filed: Mar. 17, 2010

(51) Int. Cl. A61K 38/16; A61K 38/39; A61K 38/28; A61P 17/00; A61K 31/727; A61K 38/19; A61K 39/395; A61K 39/00; A61K 38/22; A61K 38/23; A61K 38/26; A61P 7/02; A61K 48/00; A61K 31/7088; A61K 31/56; A61K 9/00; B82Y 5/00

(52) U.S. Cl. 424/400; 514/17.2; 514/5.9; 514/21.2; 514/56; 514/7.6; 424/141.1; 424/130.1; 424/184.1; 514/20.9; 514/9.7; 514/11.9; 514/11.7; 514/44 R; 514/44 A; 514/169; 977/773; 977/788; 977/906

(57) ABSTRACT

The present invention provides a method and product for transdermal delivery of therapeutics, including neurotoxins and methods for use thereof. The method and system comprises pharmaceutical compositions for facilitating transdermal delivery of therapeutics without pain by absorption and more particularly a series of pharmaceutical formulations for topical administration of neurotoxins to humans, including a neurotoxin, such as a botulinum toxin, and absorption enhancing agents that facilitate absorption of the neurotoxin through the skin of the patient and do not eliminate the bioactivity associated with the neurotoxin. The pharmaceutical compositions are topically applied on a patient, and are generally in a cream or gel form.

### FIELD OF THE INVENTION

The present invention relates to the field of drug formulations for use in conjunction with a transdermal delivery system and an improved pharmaceutical composition comprising macromolecule pharmaceuticals in micellar form for use as a component in a transdermal system for effective, pain-free transdermal delivery of therapeutics and particularly neurotoxins. The pharmaceutical compositions are particularly effective in topical applications. The present invention further relates to methods for preparing and using these pharmaceutical compositions, as well as methods for enhancing the stability and the rate of absorption of the macromolecular therapeutic agent.

### BACKGROUND OF THE INVENTION

The stratum corneum presents the most significant hurdle in transdermal delivery of medications. The stratum corneum is typically about 10-15 um thick, and it consists of flattened, keratised cells (corneocytes) arranged in several layers. The intercellular space between the corneocytes is filled with lipidic structures, and may play an important role in the permeation of substances through skin (Bauerova et al., Chemical enhancers for transdermal drug transport, European Journal of Drug Metabolism and Pharmacokinetics, 2001,26 (1/2) : 85-94). The rest of the epidermis below the stratum corneum is approximately 150 um thick. The dermis is about 1-2 mm thick and is located below the epidermis. The dermis is innervated by various capillaries as well as neuronal processes.

Most drugs are not amenable to transdermal mode of administration due to the well-known barrier properties of the skin. Molecules moving from the environment into and through intact skin must first penetrate the stratum corneum. It is the stratum-corneum that presents the greatest barrier to absorption of topical compositions or transdermally administered drugs. The stratum corneum, the outer horny layer of the skin, is a complex structure of compact keratinized cell remnants separated by lipid domains. Compared to the oral or gastric mucous, the stratum corneum is much less permeable to outside molecules.

The flux of a drug across the skin can be increased by changing either a) the resistance (the diffusion coefficient), or b) the driving force (the solubility of the drug in the stratum corneum and consequently the gradient for diffusion). Many enhancer compositions have been developed to change one or more of these factors, and are known in the art. U.S. Pat. Nos. 4,006,218, 3,551,154 and 3,472,931, for example, respectively describe the use of dimethylsulfoxide (DMSO), dimethyl formamide (DMF) and N,N-dimethylacetamide (DMA) to enhance the absorption of topically applied drugs through the stratum corneum. Combinations of enhancers consisting of diethylene glycol monoethyl or monomethyl ether with propylene glycol monolaurate and methyl laurate are disclosed in U.S. Pat. No. 4,973,468 as enhancing the transdermal delivery of steroids such as progestogens and estrogens. A dual enhancer consisting of glycerol monolaurate and ethanol for the transdermal delivery of drugs is shown in U.S. Pat. No. 4,820,720. U.S. Pat. No. 5,006,342 lists numerous enhancers for transdermal drug administration consisting of fatty acid esters or fatty alcohol ethers of C.sub.2 to C.sub.4 alkanediols, where each fatty acid/alcohol portion of the ester/ether is of about 8 to 22 carbon atoms. U.S. Pat. No. 4,863,970 shows penetration-enhancing compositions for topical application comprising an active permeant contained in a penetration-enhancing vehicle containing specified amounts of one or more cell-envelope disordering compounds such as oleic acid, oleyl alcohol, and glycerol esters of oleic acid; a C.sub.2 or C.sub.3 alkanol and an inert diluents such as water.

The use of sorbitan esters of long chain aliphatic acids as skin permeation enhancers is disclosed in U.S. Pat. Nos. 5,122,383; 5,212,199 and 5,227,169. Skin permeation enhancement using aliphatic alcohol esters of lactic acid is disclosed in U.S. Pat. No. 5,154,122, World Patent 95/09006 and in Dohi et al., Enhancing Effects of Myristyl Lactate and Lauryl Lactate on Percutaneous Absorption of Indomethacin, Chem Pharm. Bull. 38 (October 1990) 2877-2879. U.S. Pat. No. 5,314,694 also makes reference to the use of esters of fatty acid alcohols, i.e. lauryl alcohol and lactic acid as a permeation enhancer component.

World Patent 96/37231 teaches the use of acyl lactylates as permeation enhancers for drug delivery purposes. This patent is specific to esters of fatty acids and lactic acid such as caproyl lactylic acid and lauroyl lactylic acid. It is stated that the salt form of acyl lactylates are not effective as permeation enhancers.

Many of the enhancer systems possess negative side effects such as toxicity, skin irritation and incompatibility with the drugs or other ingredients making up the transdermal system. U.S. Pat. No. 4,855,294 discloses compositions for reducing skin irritation caused by drug/enhancer compositions having skin irritation properties comprising a percutaneously absorbable drug, a binary enhancer composition consisting of a solvent and a cell envelope disordering compound, combined with an amount of glycerin sufficient to provide an anti-irritating effect.

Skin permeation enhancement due to fatty acid sucrose esters is disclosed in U.S. Pat. No. 4,940,586. Penetration enhancement resulting from combining free base and acid addition salt combinations of drugs is taught in U.S. Pat. No. 4,888,354. Enhancement of drugs by means of sub-saturation in a carrier is disclosed in U.S. Pat. No. 5,164,190.

U.S. Pat. Nos. 6,066,328 and 5,658,575 “Cosmetic or dermatological composition comprising an oil-in-water emulsion based on oily globules provided with a lamellar liquid crystal coating and made of—at least one lipophilic surface-active agent, at least one hydrophilic surface-active agent, and at least one fatty acid, the coated oily globules having a mean diameter of less than 500 nanometermeters, preferably less than 200 nanometermeters, and the oily phase contains a basic agent in the dissolved state, exhibit good skin and hair penetration”

U.S. Pat. No. 6,004,566 disclose oil-in-water sub micron emulsions that enhance absorption due reduction of mean particles size to below half a micron. However, two components are needed for stabilizing the emulsion, an emulsifying stabilizer and a surfactant.

Many of the enhancer chemicals listed above were not exploited due to dose dependent skin adverse reaction that limits their clinical use. Alcohols, azones, fatty acids such as oleic acid and other suggested skin permeation enhancers are also causing skin damage or irritation or sensitization.

Transdermal administration of pharmaceuticals has been the subject of research in attempt to provide an alternative route of administration of medications without undesirable consequences associated with injections and oral delivery. For example, needles often cause localized pain, and potentially expose patients receiving injections to blood borne diseases. Oral administration suffers from poor bioavailability of medications due to the extremely acidic environment of the patient's stomach. Transdermal administration techniques attempt to overcome these shortcomings by providing non-invasive administration of pharmaceuticals. It is desirable with transdermal administration to reduce damage to a patient's skin. Thus, transdermal administration of medication may reduce or eliminate pain associated with injections, reduce the likelihood of blood contamination, and improve the bioavailability of drugs once they are incorporated systemically.

Attempts at transdermal administration of medication have attempted to improve the permeability of the stratum corneum. Most attempts of transdermal therapy are directed at administering pharmaceutical agents that are incorporated into a patient's circulatory system, and thus are systemically administered through the skin. Some attempts have included using chemical enhancing agents that increase the permeability of molecules through the skin. Some attempts have included using mechanical apparatus to bypass or ablate portions of the stratum corneum. In addition, attempts have included use of ultrasound or iontophoresis to facilitate the permeation of pharmaceuticals through the skin. As indicated above, the goal of these therapeutic methods is to deliver a pharmaceutical agent, typically a small molecule, through the skin so that an agent may pass to the capillary bed in the dermis where the agent may be systemically incorporated into the patient to achieve a therapeutic effect.

Although small molecules have been a major focus of transdermal administration techniques, it is important to note that it appears that large molecules, such as polypeptides, and protein complexes, are also amenable to transdermal administration.

The delivery of drugs to the skin and systemically via the skin and through the natural barrier of the stratum corneum is often done via creams and lotions, which are classical vehicles for delivering drugs and cosmetics to the skin. These preparations are semi-solid, bi-phasic preparations where oil spheres are dispersed in water. The droplet size of these spheres has not been a concern in conventional pharmaceutically marketed semi-solid creams and lotions. Most commercially marketed medical creams include oil spheres having a size of 5 to 50 microns. For example, VOLTAREN EMULGEL has a droplet size above five microns, as confirmed both microscopically and with photon correlation spectroscopy (Coulter N4MD).

Moreover, the scientific literature does not address the droplet size of the internal oily phase of topically applied emulsions. On the few occasions that refer to topical cream or lotion dosage forms, the indicated droplet size is in the range of a few to tens of microns. For example, U.S. Pat. No. 4,529,601 relates to an eutectic mixture of lidocaine and tetracaine which allegedly produces a good local anesthetic effect that may not be achieved otherwise.

EP 00 63 870 claims good anti-inflammatory activity and high safety of an anti-inflammatory substance in combination with MCT oil and carboxy vinyl polymer. Again, droplet size is not emphasized.

EP 04 33 132 discloses the topical cosmetic application of vesicles for incorporation of essential oils. It is also possible, according to that patent application, that small droplets of various sizes of the essential oils may be formed.

The cases cited above exemplify numerous patents concerning topical uses of classical macroemulsions, in which the oily droplets are generally well above one micron in diameter. There is also a vast body of prior art utilizing liposome preparations for enhanced dermal penetration of pharmaceuticals (Egbaria & Weiner, Adv. Drug Delivery Rev. 5, 287 (1990)). However, there are inherent problems in formulating stable liposomes, since these structures are lipid bilayers enveloping an aqueous phase. Another type of drug carrier, distinct from both classical emulsions and liposomes are the microemulsions which are usually thermodynamically stable, transparent and have particles consistently below 200 nm (Rosano, H. L., Carallo, J. L. and Lyons, G. B. Microemulsion Systems, Vol. 24, Chap. 16, H. L. Rosano and M. Clause eds. Marcel Dekker, Inc., N.Y. (1987), pp. 271). However, microemulsions contain a large proportion of surfactant to lipid and therefore are inappropriate for dermal applications due to anticipated problems of irritancy.

EP 05 06 197 discloses an aqueous suspension of nanoparticles of at least one lipid and an emulsifier, wherein the nanoparticles have a size of between 50 and 1000 nm. The lipids used therein, however, are either a solid lipid or a mixture of solid lipids.

In the field of topical and transdermal medication and delivery of drugs, much effort has been invested in providing chemical enhancers of drug penetration, such as DMSO and azones. Many of these substances cause irritation and are not desirable due to their toxicity. There remains a need, therefore, for a method and vehicle which will enable or facilitate efficient transport of poorly soluble drugs through the skin for topical or transdermal use, when provided as an aqueous dispersion of same.

Several efforts have been made to provide botulinum toxin formulations that can be stabilized and delivered in alternative ways. For example, U.S. Pat. No. 6,585,993 discloses a biocompatible implant for continuous release of a neurotoxin over a treatment period extending from one month to five years. While such an implantable system may be useful for certain situations, such as for the treatment of migraine, this type of implant system is not feasible for the treatment of facial, neck or hand wrinkles.

United States Patent Application No. 2004/0247623 suggests a method for the treatment of sensory neuron related distorters through transdermal application of a neurotoxin. This application is particularly directed to a method of treating migraine. The application suggests that botulinum toxin can be administered transdermally through a variety of ways. For example, the toxin may be incorporated into a transdermal patch or it may be administered through electrophoresis. The application also suggests that botulinum toxin can be administered using a topical cream. They teach that this would be achieved by reconstituting botulinum toxin with normal saline and then mixing the reconstituted toxin with a suitable cream or base and then massaging it on to the affected area. This type of application is unlikely to have much effect since the reconstituted botulinum toxin will have a very short active life.

International Patent Application WO 0158472 describes a pharmaceutical composition comprising botulinum toxin and a polysaccharide. This application teaches that the polysaccharide stabilizes the neurotoxin. However, other studies, as discussed above, have shown that sacharrides are poor'stabilizers for botulinum toxin.

International Patent Application WO 04/060384 discloses a pharmaceutical botulinum toxin composition which includes a sequestration agent. The purpose of the sequestration agent is to prevent the diffusion of the botulinum toxin away from the site of injection. This does not address the need for stable compositions that can be applied to the surface of the skin.

Erythropoietin, which is about 48 kD, has also been successfully transdermally administered (Mitragotri et al., Ultrasound-mediated transdermal protein delivery, Science, 1995,269: 850-853; U.S. Pat. Nos. 5,814,599; and 6,002,961).

U.S. Pat. No. 5,690,954 discloses a drug delivery system utilizing bioadhesive microspheres each having an active drug and an absorption-enhancing material. The patent does not appear to teach micelles of the size or nature of those disclosed in the present invention.

U.S. Pat. No. 5,376,646 relates to a method for facilitating penetration and distribution of a pharmaceutically active substance into the skin, which preparation includes the pharmaceutically active substance, a salt of cholanic acid, and phosphatidyl choline in an effective amount to facilitate penetration and distribution of the substance into the skin. The salt of a cholanic acid can be sodium glycocholate, and the phosphatidyl choline is lecithin.

U.S. Pat. No. 5,663,198 discloses a drug formulation with a fluorinated hydrocarbon and micronized particles of a sparingly water-soluble drug that are coated with a physiologically acceptable ampholytic phospholipid surfactant to give a micellar/colloidal solution. The phospholipid disclosed is a phosphatidylcholine.

Relatively little progress has been made in reaching the target of safe and effective non-invasive transdermal delivery of formulations for macromolecules, including peptides and proteins. Barriers to developing transdermal formulations for proteins, peptides and other large and small molecules include poor intrinsic permeability, cellular enzymatic degradation and chemical instability. Pharmaceutical approaches to address these barriers that have been successful with traditional small, organic drug molecules have not readily translated into effective peptide and protein formulations.

Various routes of administration other than injection for proteins and peptides have been explored. The ability of molecules to permeate the skin effectively appears to be related to molecular size, lipid solubility and peptide protein ionization. Molecules less than 1000 daltons appear to cross the skin barriers rapidly. As molecular size increases, the permeability of the molecule decreases rapidly. Lipid soluble compounds are more permeable than non-lipid soluble molecules. Maximum absorption occurs when molecules are un-ionized or neutral in electrical charges. Charged molecules, therefore, present the biggest challenges to absorption through the skin.

Some enhancers, especially those related to bile salts, and some protein solubilizing agents are exremely potent in transporting the molecules effectively acroos the tight junctions and skin. Several approaches have been utilized to improve the transport of the bile salt-based delivery systems, including the use of protease inhibitors and various polymer matrices. Other attempts to deliver large molecules using single bile acids or enhancing agents in combination with protease inhibitors and biodegradable polymeric materials similarly failed to achieve therapeutic levels of proteinic drugs in the patient. Single enhancing agents fail to loosen tight cellular junctions for the time needed to permit passage of large molecules through the skin membranes without further degradation.

What is needed therefore are pharmaceutical compositions or formulations containing therapeutically effective amounts of neurotoxins which enable the neurotoxin to permeate the skin of a patient and retain the neurotoxin's bioactivity to cause a therapeutic effect without undesirable pain associated with the administration of the neurotoxin.

### SUMMARY OF THE INVENTION

According to the present invention there is provided a cosmetic or pharmaceutical composition in the form of an effective amount of a macromolecular pharmaceutical agent; an alkali metal alkyl sulfate; at least one alkali metal salicylate; a pharmaceutically acceptable edetate; at least one micelle-forming compound selected from a suitable group and a suitable solvent; wherein the alkali metal alkyl sulfate, the alkali metal salicylate, the edetate and the micelle-forming compound are each present in a 1 and 20 wt./wt. %

concentration of the total composition, and the total concentration of the alkali metal alkyl sulfate, the alkali metal salicylate, the edetate and micelle-forming compound together is less than 50 wt./wt. % of the composition with the macromolecular pharmaceutical agent in micellar form.

More specifically, the present invention provides that the micelle-forming compound may be selected from the group comprising lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof, and mixtures or combinations thereof.

### BRIEF DESCRIPTION OF THE DRAWING

FIG. 1 is a photograph showing the fine line reduction of frown lines.

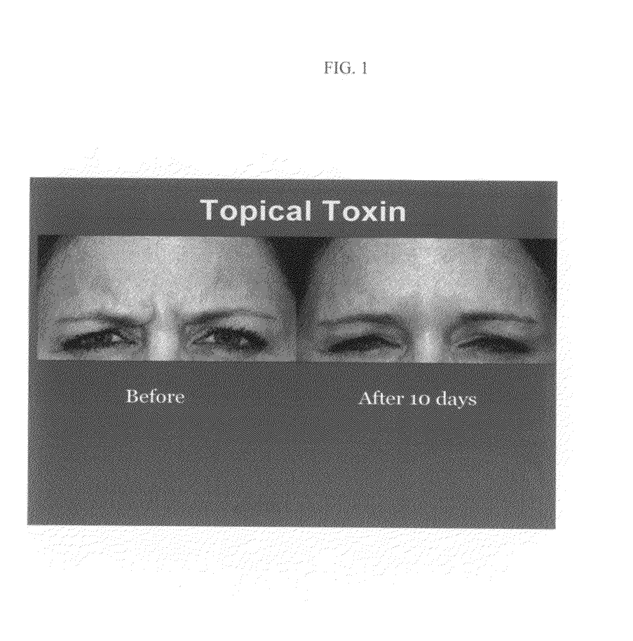

FIG. 2 is a photograph showing the fine line reduction at the subject's forehead.

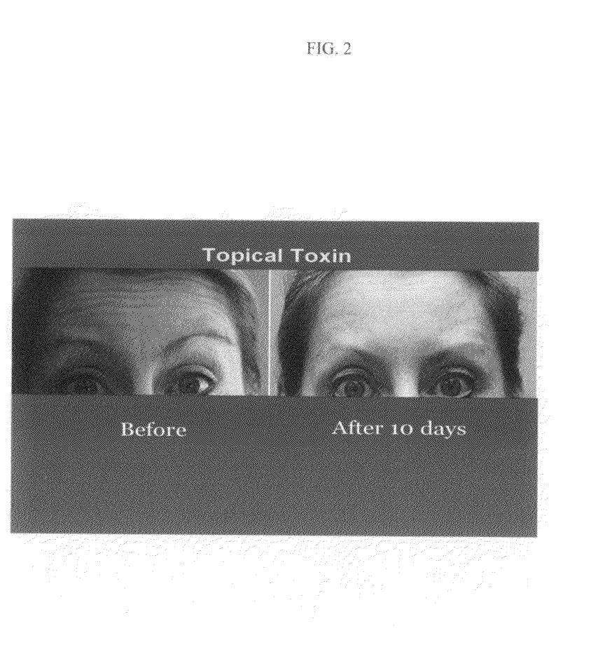

FIG. 3 is a photograph showing the fine line reduction of crows feet.

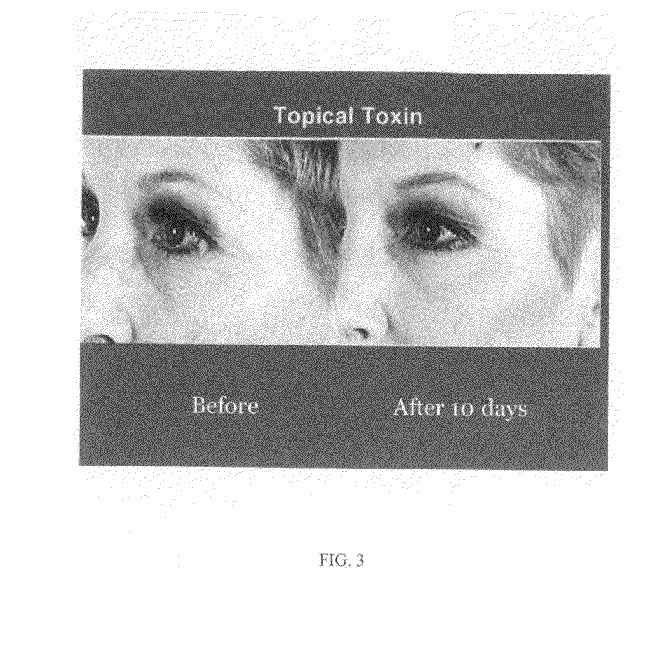

FIG. 4 is a graphic representation of the axilla and palm sweat reduction over a two week period following use an embodiment of the invention.

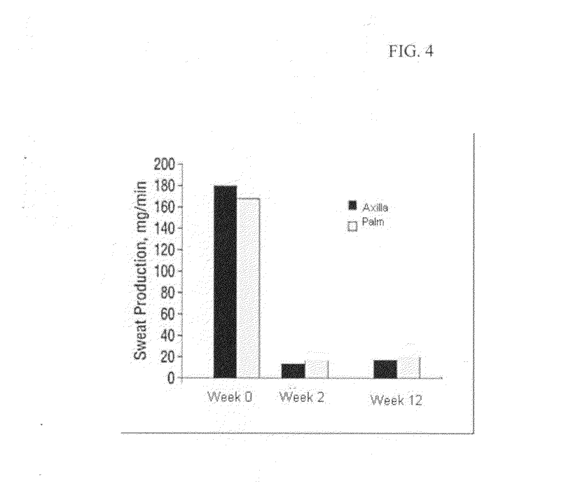

FIG. 5 is a graphic representation of the particle size distribution with regard to an embodiment of the invention.

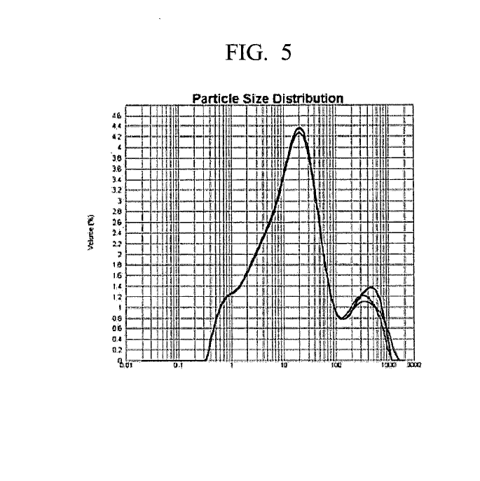

FIG. 6 are photographs of the axilla sweat reduction (before and after) after a 16 week period following use an embodiment of the invention.

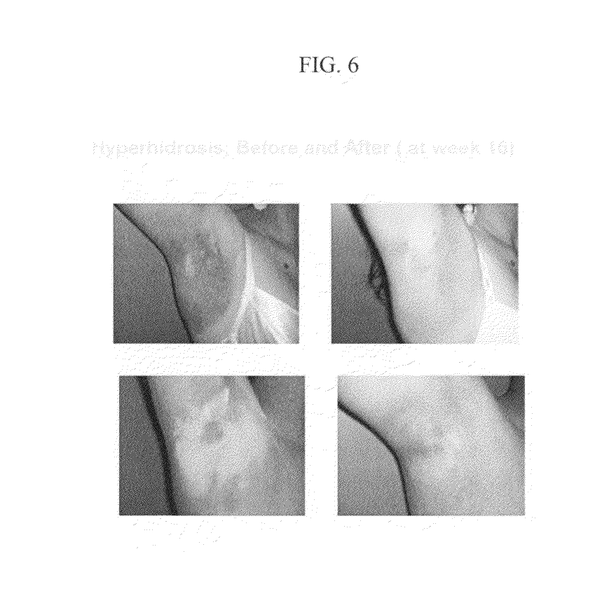

FIG. 7 are photographs of the palm sweat reduction (before and after) after a 16 week period following use an embodiment of the invention.

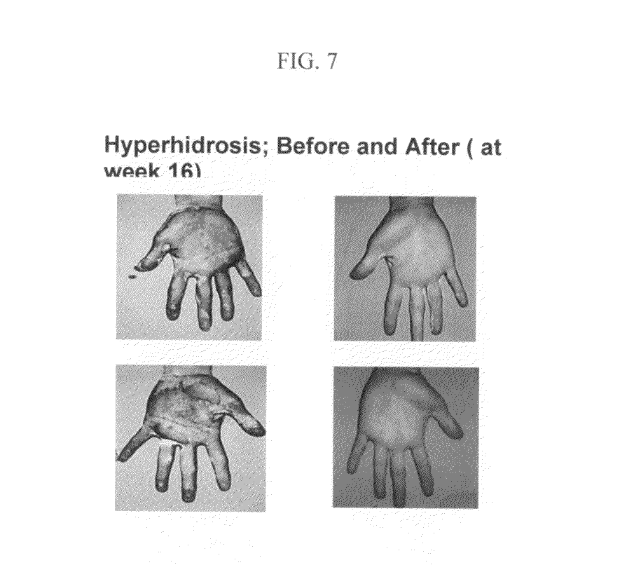

### DETAILED DESCRIPTION OF CERTAIN PREFERRED EMBODIMENTS OF THE INVENTION

### Definitions

In describing and claiming the present invention, the following terminology will be used in accordance with the definitions set forth below.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

As used herein, “formulation” and “composition” may be used interchangeably and refer to a combination of elements that is presented together for a given purpose. Such terms are well known to those of ordinary skill in the art.

As used herein, “carrier,” “inert carrier,” and “acceptable carrier” may be used interchangeably and refer to a carrier which may be combined with a plurality of nanodiamond particles in order to provide a desired composition. Those of ordinary skill in the art will recognize a number of carriers that are well known for making specific remedial healthcare and/or cosmetic compositions.

As used herein, “biologically acceptable carrier” refers to a material which is suitable for use in connection with a particular biological material. A biologically acceptable carrier is compatible with, and does not adversely affect, a biological material or subject contacted therewith under prescribed conditions.

As used herein, “cosmetic” is an adjective referring to improving the appearance of a surface or covering defects. Typically, cosmetic compositions can be used to improve aesthetic rather than functional aspects of a surface. Most commonly, cosmetic compositions are formulated for application for affecting personal appearance of the body.

As used herein, “remedial” is an adjective referring to remedying, correcting, treating, improving, or preventing an undesirable condition.

As used herein, “biological material” refers to any material which is a product of a biological organism. Typical biological materials of interest can include organic oils and the like.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10”.

“About” means approximately or nearly and in the context of a numerical value or range set forth herein means 10% of the numerical value or range recited or claimed.

“Local administration” means direct administration of a pharmaceutical at or to the vicinity of a site on or within an animal body, at which site a biological effect of the pharmaceutical is desired. Local administration excludes systemic routes of administration, such as intravenous or oral administration. Topical administration is a type of local administration in which a pharmaceutical agent is applied to a person's skin. Topical administration of a neurotoxin, such as botulinum toxin, excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

As used herein, the term “transdermal” composition includes compositions capable of passing through the stratum corneum of a subject. The term transdermal further includes compositions capable of passing through the epidermis of a subject, compositions capable of passing through the dermis of a subject, and compositions capable of passing through the hypodermis of a subject. In preferred embodiments, the term transdermal includes compositions capable of passing through the skin of a subject and reaching the underlying tissues and organs.

As used herein, the term “transdermal delivery” includes delivery of, for example, a compound through the stratum corneum of a subject. The term transdermal delivery further includes delivery of, for example, a compound through the epidermis of a subject, delivery of, for example, a compound through the dermis of a subject, and delivery of, for example, a compound through the hypodermis of a subject. In preferred embodiments, the term transdermal delivery includes delivery of, for example, a compound through the skin of a subject to the underlying tissues and organs.

“Mixed micelles” refers to at least two different types of micelles, each of which has been formed using different micelle forming compounds; for example, the present compositions comprise a mix of at least two different types of micelles—micelles formed between the pharmaceutical agent and alkali metal alkyl sulfate, and micelles formed between the pharmaceutical agent and at least one different additional micelle forming compound as disclosed herein. It will be understood that each individual micelle can be formed from more than one micelle forming compound as well. The mixed micelles of the present invention tend to be smaller than the pores of the membranes (skin). The extremely small size of the present mixed micelles helps the encapsulated macromolecules penetrate efficiently through the skin. The present compositions offer increased bioavailability of active drug, particularly across the skin, when compared with pharmaceutical preparations known in the art.

“Macromolecular”, when used in conjunction with the term pharmaceutical agent, refers to pharmaceutical agents having a molecular weight greater than about 1000 daltons; preferably the macromolecular pharmaceutical agents of the present invention have a molecular weight between about 1000 and 2,000,000 daltons although even larger molecules are also contemplated.

The macromolecular pharmaceutical agent exists in micellar form in it's intact pharmaceutical composition. A micelle is a colloidal aggregate of amphipathic molecules in which the polar hydrophilic portion of the molecules extends outwardly while the non-polar hydrophobic portion extends inwardly. The micelle encapsulates the molecule of interest. As discussed below, various combinations of micelle-forming compounds are utilized in order to achieve the present formulation. It is believed that the presence of the micelles significantly aids in the absorption of the macromolecular pharmaceutical agent both because of their enhanced absorption ability, and also because of their size. The particle size of the micelles will typically be in the range of 1 to 10 nanometers. Preferably, the micelle size ranges between 1 and 5 nanometers.

“Neurotoxin” means a biologically active molecule with a specific affinity for a neuronal cell surface receptor. Neurotoxin includes Clostridial toxins both as pure toxin and as complexed with one to more non-toxin, toxin associated proteins.

“Stabilized botulinum toxin” means a botulinum toxin that is still biologically active or that still is capable of binding to a target cell so that the botulinum toxin can effectively reduce or prevent exocytosis of intracellular molecules, such as neurotransmitters or peptides, from the cell to which the botulinum toxin is bound. Stabilized botulinum toxins are not cytotoxic.

“Enhancing agent” refers to an agent that enhances the permeability of a patient's skin so that botulinum toxin can be absorbed by the skin to achieve a therapeutic effect. In reference to the disclosure herein, enhancing agent specifically excludes dimethylsulfoxide (DMSO) or a combination of pluronic lecithin organizer (PLO) and DMSO. An enhancing agent may include, and is not limited to, alcohols, such as short chain alcohols, long chain alcohols, or polyalcohols; amines and amides, such as urea, amino acids or their esters, amides, AZONEO, derivatives of AZONE (E), pyrrolidones, or derivatives of pyrrolidones; terpenes and derivatives of terpenes; fatty acids and their esters; macrocyclic compounds; tensides; or sulfoxides other than dimethylsulfoxide, such as, decylmethylsulfoxide; liposomes; transfersomes; lecithin vesicles; ethosomes; water; surfactants, such as anionic, cationic, and nonionic surfactants; polyols; and essential oils.

The transdermal delivery system addresses the above need by providing an improved delivery of pharmaceutical compositions comprising a macromolecular pharmaceutical agents, an alkali metal alkyl sulfate, a pharmaceutically acceptable edetate, an alkali metal salicylate and at least one additional micelle-forming compound, in a suitable solvent. The agent can be one or more proteins, peptides, hormones, vaccines or drugs. The molecular weight of the macromolecular pharmaceutical agent preferably ranges between about 1,000 and 2,000,000 daltons. The agent is presented in micellar form, with a micelle size of approximately one to 10 nanometers (nm).

The transdermal delivery system also enhances the rate of absorption of a macromolecular pharmaceutical agents comprising the agent in combination with an alkali metal alkyl sulfate, a pharmaceutically acceptable edetate, at least one alkali metal salicylate, and at least one micelle-forming compound.

The transdermal delivery system is a pharmaceutical composition comprising of: an effective amount of a macromolecular pharmaceutical agent; an alkali metal alkyl sulfate; a pharmaceutically acceptable edetate; at least one alkali metal salicylate; at least one micelle-forming compound selected from the group consisting of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof, and a suitable solvent. The alkali metal alkyl sulfate, the edetate, and the alkali metal salicylate are each present in a concentration between about 1 and 20 wt./wt. % of the total composition, each micelle-forming compound concentration is between about 1 and 20 wt./wt. % of the total composition, and the total concentration of the alkali metal alkyl sulfate, edetate and the micelle-forming compound together is less than 50 wt./wt. % of the total composition.

A suitable neurotoxin used in the pharmaceutical compositions disclosed herein may be a neurotoxin made by a bacterium, for example, the neurotoxin may be made from a Clostridium botulinum, Clostridium butyricum, or Clostridium beratti. In certain embodiments of the invention, the composition may contain botulinum toxin, which may be a botulinum toxin type A, type B, type C1, type D, type E, type F, or type G. The botulinum toxin is present in the composition in an amount that results in between about 10-3 U/kg and about 10 U/kg of botulinum toxin permeating through the skin. The composition may contain an amount of botulinum toxin that causes a therapeutic effect to persist for

Most proteinic drug molecules are extremely large molecules with molecular weights exceeding 6000 daltons. In addition to being large, these molecules typically have very poor lipid solubility, and are often practically impermeable. Substances that facilitate the absorption or transport of large molecules (>1000 daltons) across biological membranes are referred to in the art as “enhancers” or “absorption aids.” These compounds include chelators, bile salts, fatty acids, synthetic hydrophilic and hydrophobic compounds, and biodegradable polymeric compounds. Many enhancers lack a satisfactory safety profile respecting irritation, lowering of the barrier function, and impairment of the mucocilliary clearance protective mechanism.

The compositions of the transdermal delivery system further comprise at least one micelle-forming compound selected from the group comprising lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof Each micelle-forming compound listed above is present in the composition in a concentration of between about 1 and 20 wt./wt. % of the total composition. More preferably, each micelle-forming compound is present in a concentration of between about 1 and 5 wt./wt. % of the total composition. The alkali metal alkyl sulfate functions as a micelle forming agent, and is added to the composition in addition to the one or more other micelle-forming compounds listed herein. The total concentration of alkali metal alkyl sulfate, the edetate and the micelle-forming compounds together is less than 50 wt./wt. % of the total composition.

The lecithin can be saturated or unsaturated, and is preferably selected from the group consisting of phosphatidylcholine, phosphatidylserine, sphingomyelin, phosphatidylethanolamine, cephalin, and lysolecithin and mixtures thereof Saturated and unsaturated lecithin are commercially available from The American Lecithin Co. as Phospholipon-H™ 0 and Phospholipon-G™, respectively.

Preferred salts of hyaluronic acid are alkali metal hyaluronates, especially sodium hyaluronate, alkaline earth hyaluronates, and aluminum hyaluronate. When using hyaluronic acid or pharmaceutically acceptable salts thereof in the present compositions, a concentration of between about 1 and 5 wt./wt. % of the total composition is preferred, more preferably between about 1.5 and 3.5 wt./wt. %.

In preferred embodiments of the transdermal delivery system, at least two micelle-forming compounds are used. The micelle-forming compound combination is selected from the group consisting of i) sodium hyaluronate and saturated phospholipid, ii) lecithin and sodium hyaluronate, iii) sodium hyaluronate and evening of primrose oil, iv) saturated phospholipid and glycolic acid, v) saturated phospholipid, glycolic acid and lactic acid, vi) sodium hyaluronate, oleic acid and gamma linoleic acid, and vii) trihydroxy oxocholanyl glycine, lecithin and chenodeoxycholate.

An isotonic agent such as glycerin or dibasic sodium phosphate may also be added after formation of the mixed micellar composition. The isotonic agent serves to keep the micelles in solution. When glycerin is used as one of the micelle-forming compounds it will also function as an isotonic agent. When dibasic sodium phosphate is used it will also serve to inhibit bacterial growth.

The pH of the present pharmaceutical composition should typically be in the range of 5 to 8, more preferably 6 to 7. Hydrochloric acid or sodium hydroxide can be utilized to adjust the pH of the composition as needed.

### EXAMPLES OF PREFERRED METHODOLOGY AND PROCESS

In another embodiment, the process comprises:

a) mixing a macromolecular pharmaceutical agent in a suitable solvent with an alkali metal alkyl sulfate, an edetate, and an alkali metal salicylate;

b) subsequently adding at least one micelle-forming compound selected from the group consisting of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof, to form a micellar macromolecular pharmaceutical agent composition; and

c) after step b), adding at least one additional micelle-forming compound which is different from that added in step b) but selected from the same group. Preferably, the micelle-forming compound selected in step b) is lecithin.

Again, during or after step b), a phenolic compound as described above can be added to the composition. Mixing can be vigorous or not. Vigorous mixing may be accomplished by using high-speed stirrers, such as magnetic stirrers, propeller stirrers, or sonicators, and is preferred.

The particle size of the micelles will typically be in the range of 1 to 10 nanometers.

Preferably, the micelle size ranges between 1 and 5 nanometers.

### EXAMPLE OF REPRESENTATIVE PREFERRED FORMULATION

| | Ingredient Name | % |
|---|---|---|
| C-CHEM9999 | WATER, RO | 29.224950 |
| C-CHEM7455 | CARBOPOL ULTREZ 10 | 0.300000 |
| C-CHEM2753 | Di Sodium EDTA | 0.100000 |
| R-CHEM0078 | SODIUM LAURYL SULFATE, Stepanol | 0.075000 |
| | WAC | |
| R-CHEM0064 | Tayl - Glycerine 99% U.S.P. | 4.000000 |
| C-MISC3342 | BUTYLENE GLYCOL | 2.000000 |
| C-CHEM2760 | TRIETHANOLAMINE 99% NF | 0.400000 |
| C-CHEM7466 | LACTIC ACID 88% | 0.200000 |
| C-CHEM2265 | Stearic Acid3X | 7.000000 |
| C-CHEM2256 | Crodacol C-95 Cetyl Alcohol | 2.000000 |
| R-CHEM0055 | Stie-Beeswax #100PNF | 0.500000 |
| C-CHEM2260 | Polawax Emulsifying Wax | 0.400000 |
| C-CHEM6506 | Liposorb L-20 | 2.500000 |
| C-OILS5560 | CEGESOFT PS6 (Vegetable Oil) | 1.000000 |
| C-OILS5544 | Evening Primrose Oil Refined (NOI) | 1.000000 |
| C-CHEM7110 | Estol 1514 isopropylmyristate | 4.000000 |
| | (viscocity adjustment) | |
| C-ESTR7119 | Octyl Palmitate (3part), Cetyl Acetate | 2.000000 |
| | (2 part), Lanolin Alcohols (3part) | |
| C-MISC3301A | SF96-350 dimethyl polysiloxane silicone | 0.250000 |
| C-MISC3298 | Sodium Hyaluronate 1% (Tri-K) | 30.000000 |
| C-CHEM3503 | Germaben II-E | 0.700000 |
| C-CHEM6252 | Crotein SPA Organic Collagen, (non- | 3.000000 |
| | animal source) | |
| C-MISC3350 | ELASTIN | 0.750000 |
| C-MISC3351 | MATRlXIL Palmitoyl Penta (or Oligo) | 0.150000 |
| | peptide | |
| C-MISC3352 | MATRIXIL 3000 Palmitoyl Oligo | 0.150000 |
| | peptide + Palmitoyl Tetra peptide | |
| C-CHEM7007 | Vitamin A Palmitate | 0.50 |
| C-CHEM7002 | Vitamin E Acetate | 0.350000 |
| C-CHEM7437 | VITAMIN B3 (NIACINAMIDE) | 0.750000 |
| C-COND2061 | LINOLEIC ACID (EMERSOL 315) | 6.000000 |
| C-315377 | Methenamine | 8.00000 |
| Add to the | 1-100 units/mL | 100.000000 |
| above Toxin-A | | |

### Representative Preferred Methodology for Above Formulation

Mix the above ingredients in one pot, cool it down to Room Temperature 20 C or so. Toxin-A is added in the required amount and then stirr the formulation at high speed 10,000 rpm for 30 minutes till very homogenous mixture is obtained. The Matrixyl and SDS with DMSO and Toxin will be added as the last step after the Toxin is mixed with them into the mixture.
Crotein is the Organic Collagen, (non-animal source)
Matrixyl and Matrixyl 3000 are pentapeptides witch will form micelles when mixed with the SDS
Trivent-Op is the mixture of Octyl Palmitate 3.00. Cetyl Acetate (and) Acetylated Lanolin Alcohols, 2.00
ESTOL 1514 is isopropylmyristate (viscocity adjustment).
CRODACOL C-95 EP conform to Cetyl Alcohol NF
   This methodology and formulation does not require the use of sphingocides and cerebrocides to encapsulate the Toxin and stabilized it. The formulation uses SDS, DMSO, EDTA and mixtures of fatty acids with Methenamine as the skin absorption enhancers.

The preferred embodiment forms micelles with Metrixyl, Metrixyl 3000, Trivent OP when mixed with SDS and DMSO along with Triethanol amine. The micelles are approximately 10 to 20 nm in size and encapsulate and stabilizes the toxin. Mixture of evening of primeose oil, vitamin A and Vitamin E oils My be employed to further stabilize the micelles by coating them.

### Transdermal Delivery of Botulinim Toxin Using Preferred Embodiment and Results of Study

Pilot Clinical Study: Aesthetics
Objective; to assess the efficacy of the Topical Toxin formulation for treatment of Glabellar, Frown lines, Crows feet and Fine lines in limited number of subject with a small dose for a period of 10 days (total dose 20 units)
Subjects: 7 (female), ages 37-61 years
Treatment: subjects were treated in the clinic with 2 units/ml topical toxin cream applied on the glabella, forehead and periorbital to treat fine lines and wrinkles (crows feet), once a day for 10 days
The topical toxin was applied on the areas with gentle rubbing and then massaging for 60-120 seconds with a novel specially designed high frequency ultrasonic vibrator
Results: The surface area occupied by deep wrinkles was reduced by a mean of 68% after 10 treatments. The surface area occupied by moderate wrinkles was reduced by a mean of 73% or more.
Glabellar lines and frown lines were reduced and showed on average 63% improvements. The results lasted for extended period of time (varied between 60 to 90 days).
The fine lines were reduced significantly under and around their eyes as seen from the photographs.
Results:
   97%—stated their overall cosmetic appearance had improved.
   77%—stated the visibility of their pores decreased.
   72%—stated the appearance of wrinkles decreased.
   63%—stated the appearance of glabellas and frown lines decreased

No product sensitivity.

### Pilot Clinical Study: Hyperhidrosis

Prospective open label study axillary and palmer hyperhidrosis
24 subjects (18-59)
Starch Iodine and Gravimetric tests
Botulinum toxin type A gel is applied, twice a day for 1 weeks, 6 units per day. (42 units at the end of the study).
Weekly follow up for 12-16 weeks, with picture records, colorimetric and gravimetric test at week 4 and at the end of the study.
Adverse effects were recorded.
Patients answered a questionnaire of satisfaction at the end of the study.
Analysis Friedman Test: mg/min: 79% reduction at 12 weeks for all areas P<0.0002
Analysis Friedman Test: cm2: 89% reduction at 12 weeks for all areas P<0.004
High Satisfaction Low AE's mostly dryness

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

What is claimed is:

1. A pharmaceutical composition comprising: an effective amount of a macromolecular pharmaceutical agent; an alkali metal alkyl sulfate; at least one alkali metal salicylate; a pharmaceutically acceptable edetate; at least one micelle-forming compound selected from the group comprised of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof, and mixtures or combinations thereof; and a suitable solvent; wherein the alkali metal alkyl sulfate, the alkali metal salicylate, the edetate and the micelle-forming compound are each present in a concentration of between about 1 and 20 wt./wt. % of the total composition, and the total concentration of the alkali metal alkyl sulfate, the alkali metal salicylate, the edetate and micelle-forming compound together is less than 50 wt./wt. % of the composition; and wherein the macromolecular pharmaceutical agent is in micellar form.

2. The composition of claim 1, wherein the alkali metal alkyl sulfate is in a concentration of from 2 to 5 wt./wt. % of the total formulation.

3. The composition of claim 1, wherein the alkali metal alkyl sulfate is an alkali metal C8 to C22 alkyl sulfate.

4. The composition of claim 3, wherein the alkali metal C8 to C22 alkyl sulfate is sodium lauryl sulfate.

5. The composition of claim 1, wherein the alkali metal salicylate is sodium salicylate.

6. The composition of claim 1, wherein each micelle-forming compound is present in a concentration of between about 1 and 5 wt./wt. % of the total composition.

7. The composition of claim 1, wherein one of the micelle forming compounds is lecithin.

8. The composition of claim 7, wherein the lecithin is either saturated or unsaturated and is selected from the group consisting of phosphatidylcholine, phosphatidylserine, sphingomyelin, phosphatidylethanolamine, cephalin, and lysolecithin and mixtures thereof

9. The composition of claim 7, further comprising an additional micelle-forming compound selected from the group consisting of hyaluronic acid, pharmaceutically acceptable salts of hyaluronic acid, and mixtures thereof, wherein the concentration of such micelle-forming compound is between about 1 and 5 wt./wt. % of the total composition.

10. The composition of claim 1, wherein said salt of hyaluronic acid is selected from the group consisting of alkali metal hyaluronates, alkaline earth hyaluronates, and aluminum hyaluronates, and the concentration of said salt is between about 1 and 5 wt./wt. % of the total composition.

11. The composition of claim 10, wherein the concentration of said salt is between about 1.5 and 3.5 wt./wt. % of said total composition.

12. The composition of claim 1, wherein at least two micelle-forming compounds are used and are selected from the group consisting of i) sodium hyaluronate and saturated phospholipid, ii) lecithin and sodium hyaluronate, iii) sodium hyaluronate and evening of primrose oil, iv) saturated phospholipid and glycolic acid, v) saturated phospholipid, glycolic acid and lactic acid, vi) sodium hyaluronate, oleic acid and gamma linoleic acid, and vii) trihydroxy oxocholanyl glycine, lecithin and chenodeoxycholate.

13. The composition of claim 1, wherein the pharmaceutical agent is selected from the group consisting of insulin, heparin, low molecular weight heparin, hirulog, hirugen, huridine, cytokines, mono and polyclonal antibodies, immunoglobins, chemotherapeutic agents, vaccines, glycoproteins, bacterial toxoids, hormones, calcitonins, glucagon like peptides, antibiotics, thrombolytic compounds, platelet inhibitors, DNA, RNA, gene therapeutics, antisense oligonucleotides, steroids and pain killers.

14. The composition of claim 1, wherein the pharmaceutical agent is insulin.

15. The composition of claim 14, wherein a first micelle-forming compound is lecithin and a second micelle-forming compound is selected from the group consisting of hyaluronic acid, pharmaceutically acceptable salts of hyaluronic acid and mixtures thereof.

16. The composition of claim 1, wherein the pH of said composition is between 5 and 8.

17. The composition of claim 1, wherein said micelle size is between about 1 and 10 nanometers.

18. The composition of claim 1, wherein said solvent is selected from the group consisting of water and ethanol.

19. The composition of claim 1 further comprising one or more of the members selected from the group consisting of a phenolic compound, an antioxidant, a protease inhibitor, and an inorganic salt.

20. The composition of claim 19 wherein said composition comprises a phenolic compound selected from the group consisting of phenol, m-cresol and mixtures thereof, in a concentration of between about 1 and 10 wt./wt. % of the total composition.

21. The composition of claim 19 wherein the antioxidant is selected from the group consisting of tocopherol, deteroxime mesylate, methyl paraben, ethyl paraben, ascorbic acid and mixtures thereof.

22. The composition of claim 19 wherein the protease inhibitor is selected from the group consisting of bacitracin, bacitracin derivatives, soybean trypsin and aprotinin.

23. The composition of claim 19 wherein the inorganic salt is selected from the group consisting of sodium, potassium, calcium and zinc salts.

24. A mixed micellar pharmaceutical composition comprising at least one alkali metal salicylate, an edetate and a macromolecular pharmaceutical agent encapsulated in micelles, said micelles formed with an alkali metal alkyl sulfate and at least one compound selected from the group consisting of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof.

25. A process for making a pharmaceutical composition comprising: a) mixing an effective amount of a macromolecular pharmaceutical agent composition in a suitable solvent with an alkali metal alkyl sulfate, an alkali metal salicylate, and an edetate; and b) slowly adding while mixing, at least one micelle-forming compound selected from the group consisting of lecithin, hyaluronic acid, octylphenoxypolyethoxyethanol, glycolic acid, lactic acid, chamomile extract, cucumber extract, ooleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof, to form a mixed micelle pharmaceutical composition; wherein the alkali metal alkyl sulfate, alkali metal salicylate, edetate and each micelle-forming compound are each present in a concentration of between about 1 and 20 wt./wt. % of the total composition, and the total concentration of alkali metal alkyl sulfate, alkali metal salicylate, edetate and micelle-forming compound together is less than 50 wt./wt. % of the total composition.

26. The process of claim 25, wherein after the addition of the micelle-forming compound of step (b) at least one additional different micelle-forming compound is mixed with the micellar composition of step (b).

27. The process of claim 25, further comprising the step of adding one or more members of the group consisting of a phenolic compound, an antioxidant, a protease inhibitor and an inorganic salt, to the mixed micelle pharmaceutical composition.

28. The process of claim 25, wherein said mixing is effected by use of a high speed stirrer selected from the group consisting of magnetic stirrers, propeller stirrers, and sonicators.

29. The process of claim 25 wherein the micelle-forming compound is formed into a film prior to the addition of the pharmaceutical agent, alkali metal alkyl sulfate, alkali metal salicylate and edetate.

30. A method for treating a patient comprising administering to said patient the pharmaceutical composition of claim 1.

32. A method for enhancing the rate of absorption of a macromolecular pharmaceutical agent in a patient comprising administering a composition comprising said agent in conjunction with an alkali metal alkyl sulfate, at least one alkali metal salicylate, an edetate and at least one micelle-forming compound selected from the group consisting of consisting of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof.

32. A pharmaceutical composition comprising: a stabilized botulinum toxin; and at least one enhancing agent for facilitating transdermal delivery of the botulinum toxin into a human patient by enhancing the permeability of the patient's skin.

33. The composition of claim 1, wherein the botulinum toxin is selected from the group consisting of botulinum toxin types A, B, Ci, D, E, F and G.

34. The composition of claim 1, wherein the botulinum toxin is botulinum toxin type A.

35. The composition of claim 1, wherein the botulinum toxin is a purified botulinum toxin.

36. The composition of claim 1, wherein the composition comprises between about 1 units to about 20,000 units of botulinum toxin.

37. The composition of claim, wherein the botulinum toxin is mixed with the enhancing agent and is applied to the patient's skin.

38. A formulation, comprising a pharmaceutical composition, which comprises: a stabilized botulinum toxin; and an enhancing agent that facilitates transdermal administration of the botuSinum toxin in a bioactive form to a subdermal target site of a human patient without being administered to the patient's circulatory system;

39. A method for making a stabilized active protein agent composition said method comprising the steps of: i. admixing sphingoside and cerebroside; ii. dissolving the admixture of step i) in alcohol; iii. removing the alcohol; iv. adding an aqueous solution of the active protein agent to form an active agent composition; and v. admixing the active agent composition with a solution of solubilized collagen and low molecular weight elastin to form a stabilized protein composition.

40. The method of claim, wherein the sphingosine and cerebroside are admixed in equal amounts.

41. The method of claim 1, wherein the alcohol is ethanol.

42. The method of claim 1, wherein the alcohol is removed by vacuum evaporation.

43. The method of claim 1, wherein the active agent composition comprises micelles.

44. The method of claim 1, wherein the solution of solubilized collagen and elastin comprises equal amounts of collagen and elastin.

45. A method for facilitating stratum corneum and dermal penetration of at least one bioactive agent having therapeutic effects comprising an effective amount of a macromolecular pharmaceutical agent; an alkali metal alkyl sulfate; at least one alkali metal salicylate; a pharmaceutically acceptable edetate; at least one micelle-forming compound and a suitable solvent; wherein the alkali metal alkyl sulfate, the alkali metal salicylate, the edetate and the micelle-forming compound are each present in a concentration of between about 1 and 20 wt./wt. % of the total composition, and the total concentration of the alkali metal alkyl sulfate, the alkali metal salicylate, the edetate and micelle-forming compound together is less than 50 wt./wt. % of the composition; and wherein the macromolecular pharmaceutical agent is in micellar form.

46. A method in accordance with claim 45, in which the micelle-forming compound is chosen from the group comprised of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof, and mixtures or combinations thereof.

47. A method in accordance with claim 45, wherein the alkali metal alkyl sulfate is in a concentration of from 2 to 5 wt./wt. % of the total formulation.

48. A method in accordance with claim 45, wherein the alkali metal alkyl sulfate is an alkali metal C8 to C22 alkyl sulfate.

49. A method in accordance with claim 48, wherein the alkali metal C8 to C22 alkyl sulfate is sodium lauryl sulfate.

50. A method in accordance with claim 45, wherein the alkali metal salicylate is sodium salicylate.

51. A method in accordance with claim 45, wherein each micelle-forming compound is present in a concentration of between about 1 and 5 wt./wt. % of the total composition.

52. A method in accordance with claim 45, wherein one of the micelle forming compounds is lecithin.

53. A method in accordance with claim 52, wherein the lecithin is either saturated or unsaturated and is selected from the group consisting of phosphatidylcholine, phosphatidylserine, sphingomyelin, phosphatidylethanolamine, cephalin, and lysolecithin and mixtures thereof.

54. A method in accordance with claim 53, further comprising an additional micelle-forming compound selected from the group consisting of hyaluronic acid, pharmaceutically acceptable salts of hyaluronic acid, and mixtures thereof, wherein the concentration of such micelle-forming compound is between about 1 and 5 wt./wt. % of the total composition.

55. A method in accordance with claim 45, wherein said salt of hyaluronic acid is selected from the group consisting of alkali metal hyaluronates, alkaline earth hyaluronates, and aluminum hyaluronates, and the concentration of said salt is between about 1 and 5 wt./wt. % of the total composition.

56. A method in accordance with claim 55, wherein the concentration of said salt is between about 1.5 and 3.5 wt./wt. % of said total composition.

57. A method in accordance with claim 45, wherein at least two micelle-forming compounds are used and are selected from the group consisting of i) sodium hyaluronate and saturated phospholipid, ii) lecithin and sodium hyaluronate, iii) sodium hyaluronate and evening of primrose oil, iv) saturated phospholipid and glycolic acid, v) saturated phospholipid, glycolic acid and lactic acid, vi) sodium hyaluronate, oleic acid and gamma linoleic acid, and vii) trihydroxy oxocholanyl glycine, lecithin and chenodeoxycholate.

58. A method in accordance with claim 45, wherein the pharmaceutical agent is selected from the group consisting of insulin, heparin, low molecular weight heparin, hirulog, hirugen, huridine, cytokines, mono and polyclonal antibodies, immunoglobins, chemotherapeutic agents, vaccines, glycoproteins, bacterial toxoids, hormones, calcitonins, glucagon like peptides, antibiotics, thrombolytic compounds, platelet inhibitors, DNA, RNA, gene therapeutics, antisense oligonucleotides, steroids and pain killers.
